(19) United States
(12) Patent Application Publication
(10) Pub. No.: US 2003/0054336 A1
(43) Pub. Date: Mar. 20, 2003

(54) METHOD FOR DETECTING HIV ANTIBODIES AND ANTIGENS USED TO THIS END

(76) Inventors: FREDERIC DONIE, PENZBERG (DE); ELKE FAATZ, HUGLFING (DE); EVA HOESS, MUNICH (DE)

Correspondence Address: BRINKS HOFER GILSON & LIONE, P. O. BOX 10395, CHICAGO, IL, 60610, US

(21) Appl. No.: 09/423,863

(22) PCT Filed: May 13, 1998
(86) PCT No.: PCT/EP98/02816

(30) Foreign Application Priority Data
May 16, 1997 (DE) 197 20 914.9

(51) Int. Cl. C12Q 1/70; C12N 9/50

(52) U.S. Cl. 435/5; 435/219

(57) ABSTRACT

The invention concerns a process of the detection of HIV antibodies against HIV by means of an immunoassay wherein at least one antigen of the env gene product gp41 of an HIV1-subtype-D isolate and at least one antigen derived from gp41 of a different HIV1-subtype of the M group is used and/or at least one antigen of gp41 of an HIV1-subtype-E isolate and at least one antigen derived from gp41 of a different HIV1-subtype isolate of the M group. The invention additionally concerns antigens and antigen mixtures with components derived from gp41 of the HIV1-subtype-D isolate and from gp41 of the HIV1-subtype-E isolate, respectively, as well as their use for the detection of HIV antibodies and a reagent kit.

The invention concerns a process of the detection of HIV antibodies against HIV by means of an immunoassay wherein at least one antigen derived from gp41 of an HIV1-subtype-D isolate, particularly from the epitope region of amino acid (AA) 518-533 of the Consensus-D sequence (=epitope region II), and at least one antigen derived from the corresponding region of gp41 of a different HIV1-subtype isolate of the M group is used and/or at least one antigen derived from gp41 of an HIV1-subtype-E isolate, particularly from the epitope region of AA 551-565 of the Consensus-E sequence (=epitope region I) and at least one antigen derived from the corresponding region of gp41 of a different HIV1-subtype isolate of the M group. The invention additionally concerns antigens and antigen mixtures with components derived from the env-gene product gp41 of the HIV1-subtype D and gp41 of HIV1-subtype E respectively as well as their use for the detection of HIV antibodies and a reagent kit.

AIDS (acquired immunodeficiency syndrome) is an acquired immunodeficiency disease caused by the HIV virus. Hitherto known pathogens are the strains HIV1 and HIV2. Both strains are similar in morphology, cell tropism, interaction with the CD4 receptor of T-cells, their in vitro cytopathic effect on CD4 cells, their general genomic structure and the capability of causing the disease AIDS (Clavel, 1987, AIDS 1, 135-140). The immunological degree of relationship is, however, only small so that generally HIV1-specific antibodies do not show any cross reactions with HIV2. Besides the most common HIV1-group-M subtypes a further HIV1 subtype, the subtype O, is known (Myers et al, Los Alamos data bank, 1994; Sharp et al. AIDS Suppl. 8, pp. 27-42, 1994). Infections with the HIV1-group M are, however, predominant.

The single HIV subtypes of the M group exhibit—despite their fundamental relationship—considerable sequence differences in some genomic regions leading partly to heterogeneities on the protein level too. It may thus for example occur that an HIV antibody detection including test components and/or antigens that specifically react with HIV1-subtype A does not react with HIV1-subtype-B samples. This leads to falsely negative test results that should in any case be avoided in the interest of the patient.

The use of peptides derived from the env region of HIV and intended for the detection of antibodies against HIV is known in the state of the art. Thus, in EP-A-0 326 490 synthetic peptides for the detection of HIV antibodies are described which are derived from the gp41 region of HIV1 and the gp36 region of HIV2 (which are there called gp42) respectively. The complete determination of antibodies against different HIV1 subtypes, in particular of the predominant group M is not possible with these peptides.

In WO 95/33206 an immunological process for the simultaneous detection of antibodies against gp41, gp36 and gag-p24 is disclosed. The process follows the bridge test principle as for example described in EP-A-0 280 211 where two antigens link the antibody to be detected. One of the antigens is bound to a solid phase. The detection of the linking antibody is obtained by the label carried by the other antigen. In the process disclosed in WO 95/33206 the peptides used as antigens are derived from gp41 (HIV1) and gp36 (HIV2) respectively and from HIV1 gag-p24. The peptide sequences for gp41 disclosed are derived from the HIV1 subtypes O and B. Further determination of subtypes ensuring that other HIV subtypes of the M group are detected reliably and with sufficient sensitivity is not possible with this process.

The problem that due to the serological heterogeneity within the HIV1-group M falsely negative results are obtained when using the hitherto known commercially available tests is described at Apetrei et al (AIDS 1996, vol. 10, pp. F57-F60). It is shown that the currently commercially available screening tests for the diagnosis of an HIV detection detect the HIV1 subtype B but non-B subtypes like for example subtype A, E or G are not detected or only slightly positive. Thus the immunological detection processes known from the state of the art have considerable disadvantages.

In the French patent application FR-A1-2 730 493 polypeptides of the glycoproteins gp 120 and gp41 derived from the HIV1 strain MAD are described. The HIV1 strain MAD presumably belongs to the group-M-subtype D. This application mentions that falsely negative detection reactions as a result of the high variability of HIV are a problem for the detection of HIV infections but does not disclose any approach to this problem.

The object of the present invention was therefore to provide an improved process for the detection of antibodies against HIV and particularly HIV1 subtypes. This improved process should ensure that especially the subtypes of the widely spread group M can be detected specifically and clearly.

This object is achieved by a process for the detection of antibodies against HIV by means of an immunoassay wherein

a) at least one antigen is derived from gp41 of an HIV1-subtype-D isolate, preferably derived from epitope region II=AA 518-533 of the Consensus D sequence and at least one antigen derived from the corresponding region of gp41 of a different HIV1-subtype isolate of the M group is used and/or

b) at least one antigen of gp41 of an HIV-1-subtype-E isolate, preferably derived from epitope region I=AA 551-565 of the Consensus E sequence and at least one antigen derived from the corresponding region of gp41 of a different HIV1-subtype isolate of the M group is used. With the aid of the process according to the invention the disadvantages of the state of the art can be mainly overcome which means that patient samples infected with HIV1 subtypes of the M group can be detected more reliably.

It has, surprisingly been revealed that with the use of antigens derived from the env-gene product gp41 and in particular from sequences of the epitope regions I and II of gp 41 of the different HIV1 subtypes a more reliable detection of HIV infections with the subtypes of the M group and subtype O can be obtained. With the process according to the invention using at least one antigen derived from gp41 of a subtype-D isolate (epitope region II=AA 518-533 of the Consensus D sequence) and/or at least one antigen derived from gp41 of a subtype-E isolate (epitope region I=AA 551-565 of the Consensus E sequence) it is guaranteed that samples containing antibodies against proteins of the group-M subtypes can now be detected more reliably than before. Also at a low antibody concentration of the sample different HIV subtypes can be detected quite well with this test. The danger of falsely negative diagnostic results can be reduced decisively by the process according to the invention.

For the process according to the invention antigens are preferably used as a mixture of different antigens. This may reduce the danger of the Hook effect in samples with high antibody concentrations since mixtures generally contain antigens with high affinities as well as antigens with low affinities. The process according to the invention also allows the use of defined antigen sequences.

The process according to the invention can be used within all procedures that are known to the expert and intended for immunological detection of HIV infections and, in particular for the detection of HIV antibodies. These involve for example homogeneous and heterogeneous immunoassays. When using the homogeneous procedure no separation of the solid and the liquid phase takes place after the reaction, i.e. after the binding of the antibody and the analyte. In homogeneous procedures the detection of the analyte can frequently be recognised by the turbidity that occurs when cross-linking of antibodies and antigens is produced by the presence of the analyte. These processes based on turbidimetric measurements are also called turbidimetric processes. In the process according to the invention the antigens used can for example be multimer antigens (polyhaptens) which are cross-linked in proportion to their concentration by the antibodies present in the sample.

Heterogeneous procedures are, however, preferred. Processes according to the bridge test and sandwich principle are for example heterogeneous processes. In the bridge test the antibody to be detected links a solid-phase bound antigen with a labeled antigen. After the immunological reaction the solid phase is separated from the liquid phase and the label determined in one of the two phases. The level of the signal is a measure of the amount or concentration of the analyte (in this case the antibody).

In the sandwich test a solid-phase bound antibody catches the analyte. A second labeled antibody also binds to the analyte. The separation of the solid and liquid phase and the detection of the label is performed analogously to the bridge test process. The procedures described should not limit the immunological detection methods possible according to the invention but should—on the contrary—clarify them. The process according to the invention is particularly preferably carried out according to the bridge test principle. Combined detection processes (called combination tests) using at the same time the bridge test for the detection of specific antibodies and the sandwich test for the detection of specific antigens are also particularly preferred. In the present case specific HIV antibodies can be detected with the aid of antigens and antigen mixtures according to the invention. When using antibodies specific for one or several HIV antigens these can be detected simultaneously in the sandwich procedure.

In the German patent application DE 197 09 762.6 a combination test is described which enables the simultaneous detection of HIV antigens and antibodies.

Within such a combination test the detection process according to the invention is preferred.

A subject matter of the patent application DE 197 09 762.6 is an immunological, preferably heterogeneous process for the detection of an HIV infection using the receptors R1 to R6. The receptors used in this process as R1 and R2 bind specifically to the HIV1-p24 and/or HIV 2-p26 antigen to be determined. The receptors used as R3 and R4 are one or more antigens from the env region of HIV1, HIV2 or HIV1-SubO (gp 160, gp120, gp41 for HIV1/HIV1-SubO and gp140, gp110, gp36 for HIV2). The receptors preferred for R3 and R4 are gp41 and/or gp36 or fragments thereof. The receptors used as R5 and R6 are one or several antigens from the pol or gag region of HIV1, HIV2 or HIV1-SubO but must not be p24 or p26. The receptors preferably used as R5 and R6 are antigens from the pol region of HIV1, HIV2 or HIV1-SubO. Particularly preferred is the use of the reverse transcriptase (RT) as receptors R5 and R6. The antigens and antigen mixtures which are described more exactly later in this application are preferably used as receptors R3 and R4 in such a combination test.

The procedures described in the German patent application DE 197 09 762.6 as well as the possibilities of solid-phase binding in a heterogeneous procedure, the processes of label detection, etc. are also components of the present application and therefore not mentioned separately here.

The process according to the invention of the present application can be performed as a wet and a dry test. In the wet tests all test reagents are present in a liquid phase. But all usual dry test formats suitable for the detection of proteins or antibodies can be used too. These dry tests or test strips as for instance described in EP-A-0 186 799 combine all test components on one single carrier. The process according to the invention is, however preferably carried out as a wet test.

All biological liquids known to the expert and probably infected with HIV can be used as samples. The samples preferred are body liquids like whole blood. blood serum, blood plasma, urine, saliva, etc.

The antigens used in the process according to the invention are derived from the env-gene product gp41 of HIV1. The antigens with sequences described in SEQ ID NO 1 to 7 which are preferably used in the process according to the invention are derived from the so-called epitope regions I and/or II of gp41 of the different HIV1 subtypes. One part of the peptides can be assigned to epitope region II; the other part to epitope region I of gp41. These regions are immunologically very reactive. A sequence comparison within these regions reveals that heterogeneities occur in the different HIV1 subtypes. It was also shown that in particular with subtype D (epitope region II) bigger sequence heterogeneities occur than with the remaining representatives of the group M.

Table 1 shows the sequence variants of different HIV1 subtypes in epitope region II of gp41, table 2 shows the sequence variants of different HIV1 subtypes in epitope region I of gp41. The figures below the sequences indicate the corresponding amino acid position in gp41.

TABLE 1
Sequence variants from the gp4l epitope region II of HIV1

| Subtype A | L | G | I | W | G | C | S | G | K | L | I | C | T | T | t | V |
|---|---|---|---|---|---|---|---|---|---|---|---|---|---|---|---|---|
| | | | | | | | | | | | | | | | n | |
| | 531 | | | | | | | | | | | | | | | 546 |
| Subtype B | L | G | I | W | G | C | S | G | K | L | I | C | T | T | a | V |
| | | | | | | | | | | | | | | | t | |
| | 547 | | | | | | | | | | | | | | | 562 |
| Subtype C | L | G | I | W | G | C | S | G | K | L | I | C | T | T | a | V |
| | | | | | | | | | | | | | | | t | |
| | | | | | | | | | | | | | | | n | |
| | 524 | | | | | | | | | | | | | | | 539 |
| Subtype D | L | G | I | W | G | C | S | G | k | H | I | C | T | T | i | V |
| | | | | | | | | | r | | | | | | t | |
| | | | | | | | | | | | | | | | n | |
| | 518 | | | | | | | | | | | | | | | 533 |
| Subtype E | L | G | L | W | G | C | S | G | K | I | I | C | T | T | A | V |
| | 562 | | | | | | | | | | | | | | | 577 |
| Subtype F | L | G | L | W | G | C | S | G | K | L | I | C | T | T | N | V |
| | 531 | | | | | | | | | | | | | | | 546 |
| Subtype G | L | G | I | W | G | C | S | G | K | L | I | C | T | T | N | V |
| | 534 | | | | | | | | | | | | | | | 549 |
| Subtype O Ant | L | S | L | W | G | C | K | G | K | L | V | C | Y | T | S | V |
| 70-isolate | | | | | | | | | | | | | | | | |
| | 581 | | | | | | | | | | | | | | | 596 |

TABLE 2
Sequence variants from the gp4l epitope region I of HIV1

| Subtype A | A | v | E | r | Y | L | r | D | Q | Q | L | L | G | I | W |
|---|---|---|---|---|---|---|---|---|---|---|---|---|---|---|---|
| | | l | | s | | | k | | | | | | | | |
| | 520 | | | | | | | | | | | | | | 534 |
| Subtype B | A | V | E | R | Y | L | k | D | Q | Q | L | L | G | I | W |
| | | | | | | | r | | | | | | | | |
| | 536 | | | | | | | | | | | | | | 550 |
| Subtype C | A | I | E | R | Y | L | K | D | Q | Q | L | L | G | I | W |
| | 513 | | | | | | | | | | | | | | 527 |
| Subtype D | A | V | E | r | Y | L | k | D | Q | Q | L | L | G | I | W |
| | | | | s | | | r | | | | | | | | |
| | 507 | | | | | | | | | | | | | | 521 |
| Subtype E | A | V | E | R | Y | L | K | D | Q | K | F | L | G | L | W |
| | 551 | | | | | | | | | | | | | | 565 |
| Subtype F | A | V | E | R | Y | L | k | D | Q | Q | L | L | G | L | W |
| | | | | | | | q | | | | | | | | |
| | 520 | | | | | | | | | | | | | | 534 |
| Subtype G | A | V | E | R | Y | L | k | D | Q | Q | L | L | G | I | W |
| | | | | | | | q | | | | | | | | |
| | | | | | | | r | | | | | | | | |
| | 523 | | | | | | | | | | | | | | 537 |
| Subtype O (Ant | A | L | E | T | L | L | Q | N | Q | Q | L | L | S | L | W |
| 70-isolate) | | | | | | | | | | | | | | | |
| | 571 | | | | | | | | | | | | | | 584 |

The positions which can be filled with several amino acids and thus contribute to the heterogeneity of the subtypes are labeled with the one-letter-amino-acid code in small letters.

The invention also concerns antigens derived from epitope region II of gp41 of the HIV1-subtype D. The antigens correspond preferably to the sequences from table 3 or partial sequences thereof it and are described by the SEQ ID NO 1 to 6 in the sequence protocol.

Table 3
Antigens from epitope region II of gp4l of HIV1-subtype D

| | SEQ | | | | | | | | | | | | | | | | |
|---|---|---|---|---|---|---|---|---|---|---|---|---|---|---|---|---|---|
| | ID NO | | | | | | | | | | | | | | | | |
| Subtype D | 1 | L | G | I | W | G | C | S | G | K | H | I | C | T | T | I | V |
| | 2 | L | G | I | W | G | C | S | G | R | H | I | C | T | T | T | V |
| | 3 | L | G | I | W | G | C | S | G | K | H | I | C | T | T | N | V |
| | 4 | L | G | I | W | G | C | S | G | R | H | I | C | T | T | I | V |
| | 5 | L | G | I | W | G | C | S | G | R | H | I | C | T | T | N | V |
| | 6 | L | G | I | W | G | C | S | G | K | H | I | C | T | T | T | V |

A further subject matter of the invention are antigens containing partial sequences of the sequences SEQ ID NO 1-6 disclosed in table 3 with a minimum length of 7 amino acids where the region contains the amino acids between the two cysteines including both cysteines. Both cysteines mostly occur in the form of an intramolecular disulfide bridge thus creating a closed ring structure, i.e. a loop. Particularly preferred are, however antigens containing partial sequences of the sequences SEQ IN NO 1-6 disclosed in table 3 with a minimum length of 10 amino acids where the region at least contains the amino acids between the two cysteines including both cysteines and three C-terminally connected amino acids. Particularly preferred partial sequences correspond to the structure given in formula (I):

C-S-G-X₁-H-I-C-T-T-X₂ (I)

X₁=K, R

X₂=I, T, N

If X=K, X₂ must not be T.

From formula (I) results the following preferred sequences SEQ ID NO 7-11 that correspond to the partial sequences preferred of SEQ ID NO 1-6:

| | | |
|---|---|---|
| SEQ ID NO 7: | C-S-G-K-H-I-C-T-T-I | (I) |
| SEQ ID NO 8: | C-S-G-K-H-I-C-T-T-N | (I) |
| SEQ ID NO 9: | C-S-G-R-H-I-C-T-T-I | (I) |
| SEQ ID NO 10: | C-S-G-R-H-I-C-T-T-N | (I) |
| SEQ ID NO 11: | C-S-G-R-H-I-C-T-T-T | (I) |

These sequences can be flanked by further amino acids or modified according to methods known to the expert. The only condition to be met is that the modified antigen must be recognized and specifically bound by the antibodies directed against the unmodified partial sequence.

A further subject matter of the invention are antigens derived from epitope region I of gp41 P1 of the HIV1-subtype E. The antigens preferably contain the sequence from table 4 or partial sequences thereof with a minimum length of 6 amino acids and are described by the SEQ ID NO 12 in the sequence protocol.

TABLE 4
Antigens from the P1 region of gp4l of HIV1-subtype E

| | SEQ ID | | | | | | | | | | | | | | | |
|---|---|---|---|---|---|---|---|---|---|---|---|---|---|---|---|---|
| | NO | | | | | | | | | | | | | | | |
| Subtype E | 12 | A | V | E | R | Y | L | K | D | Q | K | F | L | G | L | W |

With these antigens from epitope region I or II of HIV1-gp41 according to SEQ ID NO 1 to 12 a considerably improved recognition of Non-B subtypes is possible in the detection of HIV antibodies.

A further subject matter of the invention is therefore the use of an antigen according to SEQ ID NO 1 to 11 or partial sequences thereof and/or an antigen according to SEQ ID NO 12 or partial sequences thereof for the detection of antibodies against HIV, in particular of antibodies against HIV1 subtypes of the group M.

The use of antigen mixtures has proven to be particularly advantageous for a reliable detection of different HIV1 subtypes. For the detection procedure it is favorable to use at least two different antigens basing on the sequence of different subtypes. A subject matter of the invention is therefore also an antigen mixture consisting of at least two antigens with at least one antigen derived from gp41 of an HIV1 subtype D and at least one antigen from the corresponding region of gp41 of a different HIV1 subtype of the group M. The antigen of gp41 of an HIV1-subtype-D isolate preferably corresponds to the epitope region II (AA 518-533) of the Consensus D sequence where the antigen according to SEQ ID NO 1-11 is used particularly preferably.

A further subject matter of the invention is an antigen mixture consisting of at least one antigen derived from gp41 of an HIV1-subtype-E isolate and at least one antigen from the corresponding region of gp41 of a different HIV1 subtype of the group M. The antigen of gp41 of an HIV1-subtype-E isolate preferably corresponds to epitope region I (AA 551-565) of the Consensus E sequence where the antigen according to SEQ ID NO 12 is used particularly preferably. According to the invention the antigen mixture can also consist of antigens of epitope regions II and I of gp41. This mixture consists of at least one antigen derived from gp41 of an HIV1-subtype-D isolate, where an antigen of epitope region II (AA 518-533) of the Consensus D sequence is preferably used and an antigen according to SEQ ID NO 1-11 particularly preferably used, at least one antigen of the corresponding region of gp41 of a different HIV1 subtype of the group M, at least one antigen of gp41 of an HIV1-subtype-E isolate, where an antigen of epitope region I (AA 551-565) of the Consensus E sequence is preferably used and an antigen derived from SEQ ID NO 12 particularly preferably used, at least one antigen derived from the corresponding region of gp41 of a different HIV1 subtype of the group M. Such a mixture ensures great experimental safety in recognizing and detecting antibodies against different HIV1 subtypes in the gp41 region. It is, however. sufficient if the complete mixture is only provided for one epitope region (e.g. epitope region II of subtype D and a further representative of the group M) and the second region contributes only by an additional sequence (e.g. an antigen from epitope region I of only one group-M representative).

Preferably used are such antigen mixtures with an antigen from epitope region II of gp41 of an HIV1-subtype-D isolate corresponding to SEQ ID NO 1 to 6 or partial sequences thereof with a minimum length of 7 amino acids or SEQ ID NO 7 to 11 and/or with an antigen from epitope region I of gp41 of an HIV1-subtype-E isolate corresponding to SEQ ID NO 12 or partial sequences thereof with a minimum length of 6 amino acids.

Thus a further subject matter of the invention is the use of one of the antigen mixtures described above for the detection of antibodies against HIV, in particular of antibodies against HIV1 subtypes of the group M and subtype O For the simultaneous detection of subtype O the use of one or several subtype-O specific antigens from epitope regions I or II of gp41 may be necessary.

In addition to the antigen mixtures described above a further antigen derived from epitope region II of gp41 of the HIV1 subtype B and/or an additional antigen derived from the epitope region I of gp41 of the HIV1 subtype B are used particularly preferably. Such an antigen mixture again considerably improves the HIV1 subtype recognition of the group M.

In addition to the antigen mixtures described above a further antigen derived from epitope region II of gp41 of an HIV1-subtype-O isolate and/or an additional antigen derived from epitope region I of gp41 of an HIV1-subtype-O isolate are also used preferably. Such an antigen mixture again considerably improves the HIV1 subtype recognition since it ensures the recognition of group-M subtypes as well as of subtype O in one single assay. The following mixtures are given preferably as examples since they have proven to be advantageous for the determination of HIV subtypes:

Mixture of antigens derived from epitope region II of gp41:

Subtype B: L G I W G C S G K L I C T T A V

Subtype D: L G I W G C S G K H I C T T I V

Subtype O(Ant70): L S L W G C K G K L V C Y T S V

Mixture of antigens derived from epitope region I of gp 41:

Subtype E: A V E R Y L K D Q K F L G L W

Subtype B: A V E R Y L K D Q Q L L G I W

Subtype O(Ant70): A L E T L L Q N Q Q L L S L W

Additional antigens of this region recognizing further HIV subtypes like for example subtype A, C, F or G can, of course, be used too. The only condition is that the test course itself must work. If in future further HIV subtypes can be identified the expert will of course use further antigens derived from the env region of the corresponding subtype and preferably of the gp41 region.

The antigens and antigen mixtures according to the invention mentioned can of course also be used for the production of antibodies or vaccines according to methods known to the expert.

The antigens used are not only such antigens derived from HIV1 subtypes. To ensure a reliable detection of HIV infections regardless of the viral strain it is desirable to use—in addition to the antigens described of the env region of HIV1-antigens derived from the env region and especially from gp36 of HIV2.

The antigens and antigen mixtures according to the invention are—as described above-derived from an HIV1-gene product of the env region, especially of gp41 and particularly preferably from epitope region I and II of gp41. The amino acid sequence preferably corresponds to the natural sequence since this ensures the reliable detection of the different subtypes in an immunological detection process. This means that the antibodies contained in the sample and directed against a certain HIV1 subtype specifically bind to these antigens. In the case of the bridge test the antigens should be linked by the antibody.

The term antigen means a binding partner that specifically binds to an antibody with the corresponding binding site. The antigen is the epitope that is recognized and specifically bound by the antibody. The antigen preferably is a peptide or a protein and thus preferably consists of amino acids but can also be modified by sugar structures and/or lipid structures. The precondition that the antigenic properties of the antigen, i.e. the suitability for binding with the antibody to be detected are not changed considerably. The antigens can, however also be used as pure peptides without further modifications. The use of unmodified antigens is for example conceivable in competitive tests. All antigen modifications required for the corresponding procedure and known to the expert are, in principle, allowed. It is always important that the binding capacity of the antigens to the antibodies to be specifically detected is maintained.

If peptide antigens are used it can be quite advantageous to modify the peptides. This means that a peptide representing a certain epitope region can for example also have N-terminal and/or C-terminal flanking sequences that do not correspond to the specific epitope anymore. This means that the peptides may also contain non-epitope sequences that do not occur naturally in this amino acid context. The only precondition is that despite the flanking amino acids the epitopes of the peptide are preserved. This means that the antibodies to be determined specifically bind to the corresponding epitope. Moreover, it is possible to equip the peptide with spacer groups known to the expert. Here again, the only condition is that the capacity for binding to the antibodies to be determined must be preserved.

The peptides or antigens can also be modified within the epitope region, e.g. by substitution, deletion or insertion of single amino acid residue. The precondition of such modifications is, however, that the specific binding capacity of the antibodies to be determined is preserved.

The peptides and antigens according to the invention that correspond to a specific epitope of the env region and in particular of the epitope region I and II of gp41 can also be peptide derivatives with one or several amino acids derivatized by a chemical reaction. Examples of peptide derivatives according to the invention are in particular such molecules with derivatized backbone or/and reactive amino acid side groups, e.g. free amino groups, free carboxyl groups or/and free hydroxyl groups. Specific examples of amino group derivatives are sulfonamides or carboxamides, thiourethane derivatives and ammonium salts, e.g. hydrochlorides. Carboxyl group derivatives are salts, ester and amides. Examples of hydroxyl group derivatives are O-acyl or O-alkyl derivatives. The production of the peptides is preferably carried out by chemical synthesis according to methods known to the expert and needs no further explanation here. The peptides and antigens, can, in principle also be produced by means of recombinant methods where the epitopes or antigens claimed can be part of a larger recombinant protein.

The term peptide derivative also includes such peptides with one or several amino acids substituted by naturally occurring or non-naturally occurring amino acid homologues of the 20 standard amino acids. Examples of such homologues are 4-hydroxyproline, 5-hydroxylysine, 3-methylhistidine, homoserine, omithine, β-alanine and 4 aminobutanoic acid. The peptide derivatives must show an essentially equivalent binding specificity or/and affinity for the antibodies to be determined compared to the peptides or antigens from which they are derived.

Peptides or antigens according to the invention and corresponding to one specific epitope are also peptide mimetic substances, called peptide mimetics in the following, showing an essentially equivalent binding specificity or/and affinity to the antibodies to be determined like the peptides or peptide derivatives mentioned above. Peptide mimetics are compounds that are able to substitute peptides in their interaction with the antibody to be determined and to exhibit—compared to native peptides—an increased stability, especially when compared to proteinases or peptidases. Methods for the production of peptide mimetics are described by Giannis and Kolter, Angew. Chem. (applied chemistry) 105 (1993), 1303-1326 and Lee et al., Bull.Chem.Soc.Jpn. 66 (1993), 2006-2010.

The length of an epitope, i.e. the length of the antigens according to the invention is oriented towards the naturally occurring epitopes of gp41. The minimum length of an epitope usually is at least 4 to 6 amino acids. The length of choice is, however above this number, i.e. between 6 and 20 and particularly preferably between 8 and 15 amino acids. In the case of peptide mimetics or peptide derivatives an analogous molecule length or dimension is necessary.

For the use in immunoassays the antigens according to the invention can be provided with solid-phase-binding groups like biotin and haptens like digoxigenin and other label groups as, e.g. metal chelate complexes according to methods known to the expert. Methods of production of hapten labeled peptides are for example described in WO 96/03423. Processes for the production of metal-chelate labeled peptides are described in WO 96/03651.

The antigens do not only have to be used separately or as a mixture of single antigens where each antigen contains one and the same epitope only once. It can often be advantageous to have epitopes that occur more than once, i.e. as multiple epitopes. Such a multiple epitope is also called a polyhapten. Polyhaptens are especially suitable for the detection of specific IgM. In WO 96/03652 such polyhaptens and their processes of production are disclosed. The coupling of such polyhaptens to label groups, haptens and solid-phase-binding groups is disclosed there, too. The antigens according to the invention are preferably used as polyhaptens and it is easy for the expert to gather their production process from WO 96/03652.

A further subject matter of the invention is a reagent for the detection of antibodies against HIV by means of an immunoassay consisting of

a) at least one antigen derived from gp41 of an HIV1-subtype-D isolate, preferably derived from epitope region II (AA 518-533) of the Consensus-D sequence and at least one antigen derived from the corresponding region of gp41 of a different HIV1-subtype isolate of the M group and/or

b) at least one antigen derived from gp41 of an HIV1-subtype-E isolate, preferably derived from epitope region I (AA 551-565) of the Consensus-E sequence and at least one antigen derived from the corresponding region of gp41 of a different HIV1-subtype isolate of the M group and other usual test additives for immunoassays. Additional substances are for example buffers, salts, detergents and adjuvants like bovine serum albumin. The additives required are known to the expert or can be found out easily.

The invention is further described in the following examples:

### EXAMPLES

### Example 1

Synthesis of the Biotinylated Peptides

The corresponding partial sequences of the amino acid sequence of the HIV-gp41 viral protein are produced by means of fluorenyl methyl oxycarbonyl-(Fmoc)-solid-phase-peptide synthesis on a batch-peptide synthesizer, e.g. Applied Biosystems A431 or A433. For this, 4.0 equivalents each of the following Fmoc amino acid derivatives are used:

TABLE 5

| | |
|---|---|
| A | Fmoc-Ala-OH |
| C | Fmoc-Cys(Trt)-OH |
| D | Fmoc-Asp(tBu)-OH |
| E | Fmoc-Glu(tBu)-OH |
| F | Fmoc-Phe-OH |
| G | Fmoc-Gly-OH |
| H | Fmoc-His(Trt)-OH |
| I | Fmoc-Ile-OH |
| K | Fmoc-Lvs(Phenylacetyl)-OH |
| L | Fmoc-Leu-OH |
| M | Fmoc-Met-OH |
| N | Fmoc-Asn(Trt)-OH |
| P | Fmoc-Pro-OH |
| Q | Fmoc-Gln(Trt)-OH |
| R | Fmoc-Arg(Pmc)-OH |
| S | Fmoc-Ser(tBu)-OH |
| T | Fmoc-Thr(tBu)-OH |
| U | Fmoc-β-Alanine |
| V | Fmoc-Val-OH |
| W | Fmoc-Trp-OH |
| X | Boc-Lys(Fmoc)-OH |
| Y | Fmoc-Tyr(tBu)-OH |
| Z | Emoc-Aminocaproic acid |

The amino acids or amino acid derivatives are dissolved in N-methylpyrrolidone. The peptide is built up on 400 to 500 mg 4-(2′,4′-dimethoxyphenyl-Fmoc-aminomethyl)-phenoxy resin (Tetrahedron Letters 28 (1987), 2107) with a loading of 0.4-0.7 mmol/g (JACS 95 (1973), 1328). The coupling reactions for the Fmoc-amino-acid derivative are performed during 20 min with 4 dicyclohexylcarbodiimide equivalents and 4 N-hydroxy-benzotriazole equivalents in dimethylformamide as a reaction medium. After each synthesis step the Fmoc group is separated with 20% piperidine in dimethylformamide within 20 min. If the peptides contain an intramolecular disulfide bridge the Fmoc-protected peptide sequence is oxidized on the solid phase before coupling of the artificial spacer with iodine in hexafluorisopropanol/dichloromethane (Kober et al., The Peptide Academic Press, New York, 1981, pp. 145-47); subsequently the N-terminal Fmoc protective group is separated and the spacer as well as the N-terminal biotin or a bispyridyl-ruthenium complex are coupled.

The release of the peptide from the synthetic resin and the cleavage of the acid-labile protective groups—with the exception of the phenylacetyl protective group at the lysine—is carried out with 20 ml of trifluoroacetic acid, 0.5 ml ethanedithiol. 1 ml thioanisol, 1.5 phenol and 1 ml water in 40 min at room temperature. The reaction solution is subsequently mixed with 300 ml cooled diisopropylether and for complete precipitation of the peptide preserved at 0° C. for 40 min. The precipitate is filtrated, washed with diisopropylether, dissolved with a small amount of 50% acetic acid and then lyophilized. The raw material obtained is purified by preparative HPLC on Delta-PAK RP C18 material (column 50×300 mm, 100 Å, 15μ) above a corresponding gradient (eluent A: water, 0.1% trifluoroacetic acid, eluent B: acetonitrile, 0.1% trifluoroacetic acid) in 120 min. The identity of the material eluted is checked by means of ionic spray mass spectrometry.

TABLE 6
Synthesized biotinylated peptides:

| | | | | | | | | | | | | | | | | | | | |
|---|---|---|---|---|---|---|---|---|---|---|---|---|---|---|---|---|---|---|---|
| Consensus B | Antigen 1 | Biotin | XUZ | L | G | I | W | G | C(ox) | S | G | K | L | I | C(ox) | T | T | A | V |
| | | | U | | | | | | | | | | | | | | | | |
| Consensus D | Antigen 2 | Biotin | XUZ | L | G | I | W | G | C(ox) | S | G | K | H | I | C(ox) | T | T | I | V |
| | | | U | | | | | | | | | | | | | | | | |

The name “C (ox)” represents an intramolecular disulfide bridge . . . XUZU “is the spacer (see table 5).

### Example 2

Synthesis of the Digoxigenylated Peptides

The peptide synthesis is performed analogously to example 1. If lysine is in the sequence the amino acid derivative Fmoc-Lys(PhAc)-OH is used for synthesis instead of Fmoc-Lys(Boc)-OH. Synthesis is completed after cleaving of the N-terminal Fmoc protective group of the last spacer amino acid. During the release of the peptide from the synthetic resin and the cleavage of acid-labile protective groups the phenylacetyl protective group is not removed from the lysine.

The introduction of the digoxigenin or digoxin label is carried out by an active ester derivative (e.g. digoxigenin-3-carboxymethylether-N-hydroxysuccinimide ester) to the free amino groups of the peptide in solution. The peptide to be derivatized is dissolved in a mixture of DMSO and 0.1 M potassium phosphate buffer, pH 8.5. Subsequently, 2 equivalents of active ester dissolved in a small amount of DMSO are added dropwise per free primary amino function and agitated at room temperature. The reaction progress is observed by analytical HPLC. The product is purified by means of preparative HPLC.

If the peptide contains lysines still protected with phenylacetyl this protective group is in the last step enzymatically separated with immobilized PenG-amidase in aqueous medium with organic solvent portion at room temperature. The immobilized enzyme is filtrated and the peptide purified by preparative HPLC. The identity of the material eluted is checked by means of ionic spray mass spectrometry.

TABLE 7
Digoxigenylated peptides:

| | | | | | | | | | | | | | | | | | | | |
|---|---|---|---|---|---|---|---|---|---|---|---|---|---|---|---|---|---|---|---|
| Consensus B | Antigen 3 | Dig-3-cme- | UZU | L | G | I | W | G | C(ox) | S | G | K | L | I | C(ox) | T | T | A | V |
| Consensus D | Antigen 4 | Dig-3-cme- | UZU | L | G | I | W | G | C(ox) | S | G | K | H | I | C(ox) | T | T | I | V |

### Example 3

Evaluation of the Subtype-specific Antigens

3.1 Immunological Test in General

The test is performed analogously to the procedure described in the instructions for use of the Enzymun tests® anti-HIV1+2+SubtypeO (ref. no. 1557319, Boehringer Mannheim GmbH, Germany). Only the antigen flasks 2a and 2b were substituted by a peptide solution (amount of antigens used was 5 nmol/ml each). The buffers and detection reagents were not changed. The test is performed at 25° C. on the instrument ES600 or ES700 (manufacturer: Boehringer Mannheim GmbH, Germany) in a sample volume of 100 μl in streptavidin-coated test tubes according to the principle of the 2-step-sandwich ELISA. The following reagents were used:

Incubation buffer: Tris 50 mM pH 7.5; bovine serum components

Conjugate buffer: Tris 50 mM pH 7.5; bovine serum components

Conjugate: Peroxidase-(POD) labeled sheep anti-digoxigenin antibodies

Substrate: ABTS® substrate solution (2.2′azino-di[3-ethylbenzthiazoline sulfonate] 1.9 mmol/l in 100 mmol/l phosphate/citrate buffer, pH 4.4, sodium perborate 3.2 mmol/l

3.2 Evaluation Results

Flask 2a

| | | |
|---|---|---|
| Antigen 1 | Antigen 2 | Antigen 1 + 2 |
| (Subtype B) | (Subtype D) | (Subtype B + D) |

Flask 2b

| | | Antigen 3 | Antigen 4 | Antigen 3 + 4 |
|---|---|---|---|---|
| Serum | Subtype | (Subtype B) | (Subtype D) | (Subtype B + D) |
| Negative control | all signals in | 0.05 | 0.03 | 0.05 |
| | U | | | |
| Serum 1 | B | 4.49 | 4.10 | 4.55 |
| Serum 1 dilution 1:10 | | 4.10 | 3.24 | 4.32 |
| Serum 1 dilution 1:100 | | 2.78 | 0.56 | 3.06 |
| Serum 1 dilution 1:1000 | | 2.10 | 0.15 | 2.28 |
| Serum 1 dilution 1:10000 | | 0.69 | 0.05 | 0.74 |
| Serum 1 dilution 1:100000 | | 0.17 | 0.03 | 0.18 |
| Serum 1 dilution 1:1000000 | | 0.05 | 0.01 | 0.05 |
| Serum 2 | D | 3.45 | 4.75 | 4.82 |
| Serum 2 dilution 1:10 | | 2.71 | 4.24 | 3.97 |
| Serum 2 dilution 1:100 | | 2.19 | 3.98 | 2.94 |
| Serum 2 dilution 1:1000 | | 1.49 | 2.55 | 2.63 |
| Serum 2 dilution 1:10000 | | 0.37 | 1.04 | 0.77 |
| Serum 2 dilution 1:100000 | | 0.16 | 0.20 | 0.29 |
| Serum 2 dilution 1:1000000 | | 0.03 | 0.09 | 0.06 |
| Serum 3 | B | 3.07 | 2.58 | 3.08 |
| Serum 3 dilution 1:10 | | 2.39 | 0.35 | 2.46 |
| Serum 3 dilution 1:100 | | 1.74 | 0.12 | 1.87 |
| Serum 3 dilution 1:1000 | | 0.58 | 0.08 | 0.69 |
| Serum 3 dilution 1:10000 | | 0.16 | 0.05 | 0.20 |
| Serum 3 dilution 1:100000 | | 0.04 | 0.04 | 0.05 |
| Serum 3 dilution 1:1000000 | | 0.00 | 0.02 | 0.00 |
| Serum 4 | B | 5.08 | 4.99 | 4.67 |
| Serum 4 dilution 1:10 | | 4.68 | 4.72 | 4.89 |
| Serum 4 dilution 1:100 | | 2.89 | 2.86 | 3.35 |
| Serum 4 dilution 1:1000 | | 3.04 | 0.85 | 3.07 |
| Serum 4 dilution 1:10000 | | 1.29 | 0.15 | 1.38 |
| Serum 4 dilution 1:100000 | | 0.62 | 0.05 | 0.69 |
| Serum 4 dilution 1:1000000 | | 0.40 | 0.00 | 0.46 |

The use of the antigen mixture containing antigens of different HIV subtypes has proven to be advantageous when compared with the use of the single antigens: the use of the antigen mixture leads to a considerably earlier recognition (with higher dilution) of infected samples. i.e. by using an antigen mixture the dilution sensitivity is influenced in a positive way. With the antigen mixtures according to the invention infections with subtype B and subtype D can be detected more reliably. Subtype B sera react with subtype-B specific antigens in higher dilutions than with subtype-D specific antigens. Analogously, subtype D sera react positively with subtype-D specific antigens also in higher dilutions whereas they react less with subtype-B specific antigens.

1. Process for the detection of antibodies against HIV by means of an immunoassay wherein
a) at least one antigen of gp24 of an HIV1-subtype-D isolate and at least one antigen derived from gp41 of a different HIV1 subtype of the group M is used and/or
b) at least one antigen of gp24 of an HIV1-subtype-E isolate and at least one antigen derived from gp41 of a different HIV1 subtype of the group M is used.

2. Process as claimed in claim 1 wherein
a) at least one antigen derived from epitope region II of the Consensus sequence of an HIV1-subtype-D isolate and at least one antigen derived from the corresponding region of gp41 of a different HIV1 subtype of the M group is used and/or
b) at least one antigen from epitope region I of the Consensus sequence of an HIV-1-subtype-E isolate and at least one antigen derived from the corresponding region of gp41 of a different HIV1 subtype of the M group is used.

3. Process as claimed in claim 1 wherein the antigen of gp41 of an HIV1-subtype-D isolate corresponds to SEQ ID NO 1 to 11 or partial sequences thereof
and/or
the antigen of gp41 of an HIV1-subtype-E isolate corresponds to SEQ ID NO 12 or partial sequences thereof.

4. Antigen mixture consisting of at least two antigens with at least one antigen derived from gp41 of an HIV1-subtype-D isolate and at least one antigen of gp41 of a different HIV1 subtype of the group M
and/or
at least one antigen derived from gp41 of an HIV1-subtype-E isolate and at least one antigen of gp41 of a different HIV1 subtype of the group M.

5. Antigen mixture as claimed in claim 4 wherein
the antigen of gp41 of an HIV1-subtype-D isolate is derived from epitope region II of the Consensus sequence of HIV1-subtype D
and/or
the antigen of gp41 of an HIV1-subtype-E isolate is derived from epitope region I of the Consensus sequence of HIV1-subtype E.

6. Antigen mixture as claimed in claim 4 or 5 wherein
the antigen of gp41 of an HIV1-subtype-D isolate corresponds to SEQ ID NO 1 to 11 or partial sequences thereof with a minimum length of 7 AA,
and/or
the antigen of gp41 of an HIV1-subtype-E isolate corresponds to SEQ ID NO 12 or partial sequences thereof with a minimum length of 6 AA.

7. Antigen mixture as claimed in one of the claims 4 to 6 wherein an additional antigen is used that is derived from epitope region I and/or II of HIV1-subtype O.

8. Antigen containing a sequence according to SEQ ID NO 1 to 11 or partial sequences thereof with a minimum length of 7 AA.

9. Antigen containing a sequence according to SEQ ID NO 12 or partial sequences thereof with a minimum length of 6 AA.

10. Use of an antigen mixture as claimed in one of the claims 5 to 7 for the detection of antibodies against HIV.

11. Use of an antigen as claimed in one of the claims 8 or 9 for the detection of antibodies against HIV.

12. Use of an antigen as claimed in claim 8 or 9 or of an antigen mixture as claimed in one of the claims 5 to 7 in a combination test according to DE 197 09 762.6 for the detection of antibodies against HIV.

13. Reagent for the detection of antibodies against HIV by means of an immunoassay consisting of
a) at least one antigen of gp24 of an HIV1-subtype-D isolate and at least one antigen derived from gp41 of a different HIV1 subtype of the group M
and/or
b) at least one antigen of gp24 of an HIV1-subtype-E isolate and at least one antigen derived from gp41 of a different HIV1 subtype of the group M and the usual test additives for immunoassays.

14. Reagent for the detection of antibodies against HIV by means of an immunoassay consisting of
a) at least one antigen of gp41 of an HIV1-subtype-D isolate from epitope region II of the Consensus sequence of HIV1-subtype D and at least one antigen derived from gp41 of a different HIV1subtype of the M group and/or
b) at least one antigen of gp41 of an HIV1-subtype-E isolate from epitope region I of the Consensus sequence of HIV1-subtype E and at least one antigen derived from gp41 of a different HIV1 subtype of the M group and the usual test additives for immunoassays.
